(19) United States
(12) Patent Application Publication
(10) Pub. No.: US 2024/0017260 A1
(43) Pub. Date: Jan. 18, 2024

(54) TESTING DEVICES, SYSTEMS AND METHODS

(71) Applicant: LEX DIAGNOSTICS LTD, Royston (GB)

(72) Inventors: Justin Rorke Buckland, Comberton (GB); Hoi Kei Chiu, Cambridge (GB); Amelia Serena Kristelle Fernandez, Cambridge (GB); Amaru Daniel Araya-Williams, Cambridge (GB)

(21) Appl. No.: 18/252,573

(22) PCT Filed: Nov. 10, 2021
(86) PCT No.: PCT/EP2021/081306

§ 371 Date: May 11, 2023

(30) Foreign Application Priority Data
Nov. 11, 2020 (GB) 2017779.6

(51) Int. Cl. B01L 3/00

(52) U.S. Cl. CPC B01L 3/5029; B01L 2200/026; B01L 2300/161; B01L 2300/0681; B01L 2300/18; B01L 2200/0684

(57) ABSTRACT

Testing devices for testing a swab sample, the devices including a swab chamber configured to receive the swab sample; an elution source configured to supply an eluent to the swab chamber, wherein the eluent is a liquid that interacts with the swab sample to produce an eluate indicative of a property of the swab sample; a discard channel configured to receive a first portion of the eluate from the swab chamber to be discarded; an analysis channel configured to receive a second portion of the eluate from the swab chamber to be processed; and a channel selector configured to selectively direct the first portion into the discard channel and the second portion into the analysis channel.

### CROSS REFERENCE TO RELATED APPLICATIONS

This application claims priority under 35 U.S.C. § 119 from British Patent Application No. GB2017779.6, filed on Nov. 11, 2020, the contents of which is incorporated herein by reference in its entirety.

### TECHNICAL FIELD

The disclosure relates to testing devices for processing or analysis of biological samples. Additionally, the disclosure relates to techniques for eluting and analysing a biological sample obtained using a swab.

### BACKGROUND

Currently, swab testing is typically done in multiple discrete stages.

First, the swab sample is converted into a more flexible form for testing, by eluting the swab to produce a liquid sample. Then, the liquid sample may be subjected to one or more stages of processing to produce an analyte, which is then finally measured to produce a test result.

For example, in the case of virus testing, the liquid sample may be lysed to break down virus particles and to release nucleic acids in the sample, and then PCR may be performed to amplify the viral nucleic acid content of the sample.

These discrete stages can be time and labour intensive, with each stage of the swab testing requiring a technician to transfer a partially-processed sample between different equipment. This also poses an exposure risk where the analyte is infectious.

Accordingly, it is desirable to provide a way of automating at least two stages of the swab testing process in a single apparatus.

### SUMMARY

Simply connecting an elution apparatus to a liquid sample processing apparatus does not give optimal sample testing properties. Specifically, the properties of the eluate change over time as the swab sample is washed, such that it is advantageous to select a specific fraction of the eluate for analysis.

According to a first aspect, the present disclosure provides testing devices for testing a swab sample, wherein the devices include: a swab chamber configured to receive the swab sample; an elution source configured to supply an eluent to the swab chamber through a chamber inlet of the swab chamber, wherein the eluent is a liquid that interacts with the swab sample to produce an eluate indicative of a property of the swab sample; a discard channel configured to receive a first portion of the eluate from a chamber outlet of the swab chamber to be discarded; an analysis channel configured to receive a second portion of the eluate from the chamber outlet to be processed; and a channel selector configured to selectively direct the first portion into the discard channel and the second portion into the analysis channel.

Optionally, the chamber inlet is arranged closer to a tip of a swab head than a break point of the swab head and the chamber outlet is located farther from the tip of the swab head than the break point of the swab head, so that the eluent impinges initially on the tip of swab head and flows across the swab head to the chamber outlet.

Optionally, the chamber inlet comprises a feature that prevents a swab from sealing the inlet.

Optionally, the elution source includes a compressible external surface of the testing device. With this configuration, driving of the eluent can be controlled mechanically from outside the device, and the structure of the device can be simplified.

Optionally, the channel selector is configured to direct the first portion of the eluate into the discard channel before directing the second portion of the eluate into the analysis channel. With this configuration, the testing device can be used with a sample that requires some initial washing before the eluate reaches a most useful composition for analysis.

Optionally, the channel selector includes a junction between the discard channel, the analysis channel and a chamber outlet from the swab chamber. This configuration provides a simple passive means for channel selection.

Optionally, in the junction, an angle to redirect flow from the chamber outlet to the analysis channel is greater than an angle to redirect flow from the chamber outlet to the discard channel. This configuration provides a simple passive means for channel selection.

Optionally, the chamber outlet and the discard channel are arranged substantially opposite each other across the junction, and the analysis channel is arranged substantially at a right angle to the chamber outlet around the junction.

This configuration provides a simple passive means for channel selection.

Optionally, the analysis channel includes a hydrophobic surface. This configuration provides a simple passive means for channel selection.

Optionally, the discard channel and/or the analysis channel includes an air outlet and a hydrophobic filter arranged at the air outlet, such that the discard channel and/or the analysis channel can be initially filled with air, and the eluate replaces the air. This configuration provides a mechanism for allowing a controlled volume of eluate to flow in the discard channel and/or the analysis channel, without creating a risk of a leak of the eluate from the testing device and risk of exposure where the analyte is infectious.

Optionally, the air outlet is connected to a closed air chamber. By including a closed air chamber, increasing air pressure can be used to assist in controlling flow of the eluate and can further reduce the risk of a leak of the eluate from the testing device.

Optionally, the analysis channel includes a lysing section. This enables the testing device to perform multi-stage analysis of a swab sample in a single device.

Optionally, the analysis channel includes an amplification section for amplifying an analyte that is to be detected in the eluate. This enables the testing device to perform multi-stage analysis of a swab sample in a single device.

Optionally, the testing device is a disposable cartridge. By providing the device as a disposable cartridge, samples can be analysed in a clean, replaceable environment.

Optionally, an opening of the swab chamber in the testing device is narrower in cross-sectional area or diameter than a nominal maximum radius of a swab head.

According to a second aspect, the present disclosure provides sample analysis systems for testing a swab sample, wherein the systems include: a disposable cartridge as described herein; and an operating module for operating the disposable cartridge to test the swab sample, wherein the operating module is configured to receive or engage with the disposable cartridge, and wherein at least one of: the operating module includes a driver configured to drive the eluent from the eluent source to the swab chamber; or the disposable cartridge includes a heater for supplying heat to a location in the analysis channel, and the operating module includes a heater driver arranged to provide electrical drive to the heater; or the operating module includes a heater for supplying heat to a location in the analysis channel.

The sample analysis systems of the second aspect have the advantage that complete analysis can be performed on multiple samples at minimal cost and preparation time, by performing the analysis within a disposable cartridge, and by retaining reusable parts of the analysis system in an operating module separate from the disposable cartridge.

According to a third aspect, the present disclosure provides testing methods for testing a swab sample, wherein the methods include: arranging a swab sample in a swab chamber; supplying an eluent to the swab chamber, wherein the eluent is a liquid that interacts with the swab sample to produce an eluate indicative of a property of the swab sample; directing a first portion of the eluate from the swab chamber to a discard channel to be discarded; and directing a second portion of the eluate from the swab chamber to an analysis channel to be processed.

By selecting a preferred fraction of the eluate, subsequent sample analysis can be performed more efficiently.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic block diagram of a disposable cartridge according to an embodiment of the present disclosure.

FIGS. 2A and 2B are graphs of PCR amplification curves obtained using different fractions of an eluate.

FIG. 3 is a schematic top view of a specific embodiment of the disposable cartridge described herein.

FIGS. 4A to 4E are schematic illustrations of steps in a process of eluting and lysing a sample using a disposable cartridge according to the embodiment of FIG. 3.

FIG. 5 is a schematic diagram of a sample analysis system in which the cartridges described herein can be used.

FIG. 6 is an illustration of a swab chamber for a disposable cartridge as described herein.

FIGS. 7A to 7C are schematic illustrations of a fluid inlet of a swab chamber as described herein.

FIG. 8 is a schematic illustration of another swab chamber embodiment for a disposable cartridge as described herein.

### DETAILED DESCRIPTION

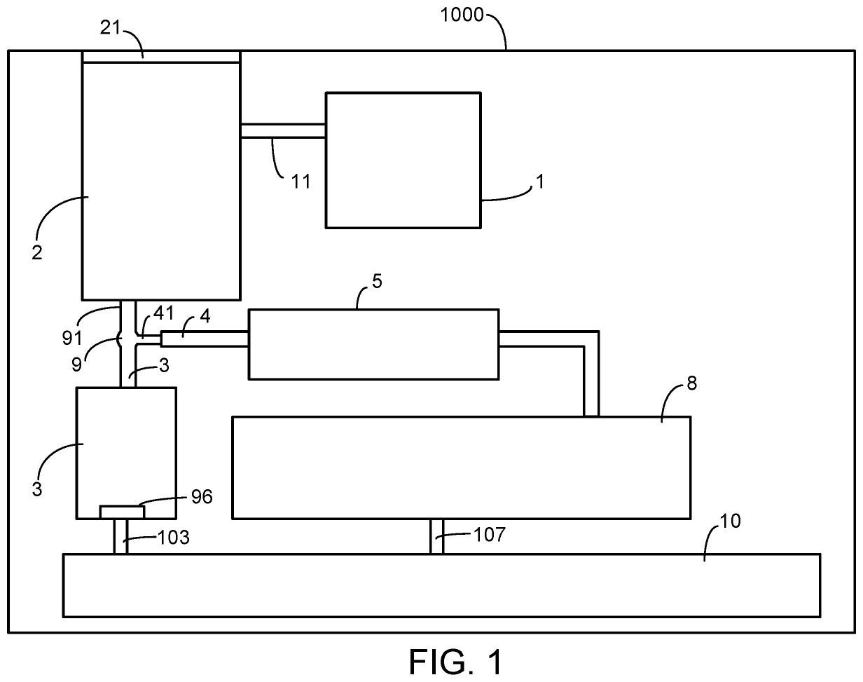

FIG. 1 schematically illustrates block features of a disposable cartridge configured for performing multiple stages of analysing a swab sample.

The disposable cartridge is an example of a testing device that is designed to work with an operating module. The operating module comprises relatively expensive and reusable parts such as system drive and control electronics, mechanical actuators and optical sensors, while the disposable cartridge deals with the direct handling of the sample. This means that the operating module does not need to be cleaned between tests, while the disposable cartridge cost is kept low, meaning that tests can be performed efficiently and at low cost. However, in other embodiments, the below-described system for integrated multi-stage testing may be embodied in a reusable form, in a single device that is cleaned between tests.

The general structure of the cartridge 1000 can be constructed from any suitable material, such as plastics, for example propylene.

The cartridge, for example, has a substantially planar layout such that internal structures of the cartridge 1000 can interact with the operating module, for example to receive heating by the operating module, to allow cooling, to provide electrical contact with components of the cartridge 1000, or to allow optical detection of an analyte through a wall of the cartridge 1000.

The cartridge 1000 comprises a swab chamber 2 configured to receive the swab sample (not shown).

The swab chamber 2 comprises an opening configured to receive the swab sample. The swab chamber 2 may also have a lid 21 configured to seal the swab chamber 2. Alternatively, the swab itself may comprise a sealing component to seal the opening, and the lid 21 may be omitted.

When the swab sample is in the swab chamber 2, the swab chamber is sealed and an eluent is run through the swab chamber 2. The swab chamber 2 comprises an inlet 11 and an outlet 91 for the elution, and the chamber 2, for example, is configured to fit closely around a swab of known size, so that the eluent flows through or closely around the swab. Many swabs are partly compressible. For such swabs, the swab chamber may be configured to have a cross-sectional area that is smaller than the uncompressed swab, so that the swab is partly compressed in the swab chamber 2 and the eluent is forced to pass through the swab to pick up more of the sample.

A position of the inlet 11 and outlet 91 may be chosen to control what proportion of the swab is eluted. More specifically, the swab chamber 2 may be elongate and configured to receive an elongate swab. By positioning the inlet and outlet closer together or further apart along the length of the chamber 2, a sample size may be effectively reduced or enlarged by only eluting part of the swab.

The cartridge 1000 further comprises an elution source 1 configured to supply an eluent to the swab chamber 2. The eluent is a liquid that interacts with the swab sample to produce an eluate indicative of a property of the swab sample. The specific eluent is chosen according to the type of sample being analysed, such that the required components of the swab sample dissolve in or mix with the eluent.

In some embodiments, the eluent is stored in the elution source 1, and the elution source 1 comprises a compressible external surface of the disposable cartridge. This enables driving of the eluent through the swab chamber 2 by means of compressing the elution source 1. For example, the separate operating module may comprise a piston for compressing the elution source 1. The eluent may instead be driven by other means such as a pump within the disposable cartridge. As a further alternative, the elution source 1 may by adapted to connect with an external eluent source, such that the cartridge is not required to initially contain a supply of the eluent, and the cartridge need not be capable of driving the eluent on its own.

The cartridge 1000 further comprises a discard channel 3 configured to receive a first portion of the eluate from the swab chamber, an analysis channel 4 configured to receive a second portion of the eluate from the swab chamber. In one specific tested example, the first portion is the first 50 microliters of eluate that flows through the outlet 91, and the second portion is the next 40 microliters of eluate that flows through the outlet 91.

For example, the discard channel 3 and the analysis channel 4, as well as the chamber inlet 11 and outlet 91, comprise relatively narrow channels in which the eluent/eluate experiences relatively simple capillary flow. The discard channel 3 and analysis channel 4 may additionally comprise one or more chambers configured to fill with eluate.

Additionally, a channel selector 9 is configured to selectively direct the first portion of the eluate into the discard channel 3 and the second portion into the analysis channel 4.

In other words, the channel selector 9 is used to select a fraction of the eluate for analysis.

For example, in the specific case of a nasal swab sample that comprises mucin and target material for analysis such as virus particles or cells, the mucin may be washed off the swab sample more easily than the target material, such that initial fractions of the eluate have a higher ratio of mucin to target material, and later fractions have a lower ratio of mucin to target material. In the case where the desired test is to determine if the target material contains a specific virus, then it is desirable to discard initial fractions of the eluate, with lower target to mucin concentration ratio, and to analyse later fractions of the eluate, with higher target to mucin concentration ratio. A later fraction may contain reduced target material concentration and a relatively more reduced mucin concentration than an earlier fraction. This may allow target material to be detected more readily in the later fraction, as the relatively higher mucin concentration in the earlier fraction may inhibit the detection process.

As another example, the initial eluate portion is more likely than the later portion to contain entrained air or bubbles interspersed with the eluted liquid, and such air can be problematic for subsequent analysis. Therefore, a further benefit of discarding the initial portion of eluate is that the analysis portion of eluate is more likely to be free of entrained air or bubbles.

For example, for simplicity of construction and operation, the channel selector 9 is implemented as a passive selector using the shape of a junction between the discard channel 3, the analysis channel 4 and the chamber outlet 91.

For example, if an angle to redirect flow from the chamber outlet 91 to the analysis channel 4 is greater than an angle to redirect flow from the chamber outlet 91 to the discard channel 3, then the momentum of the fluid eluate will carry the eluate into the discard channel so long as the discard channel 3 is not filled. Once the discard channel 3 is filled, then liquid pressure at the junction will redirect further eluate into the analysis channel 4.

It is noted that, under this simple construction, some eluate may flow into the analysis channel 4 before the discard channel 3 is filled. Nevertheless, there is still a benefit to selecting the second portion of the eluate, even if the separation of the first and second portions of the eluate is not perfect, and therefore a small proportion of the first portion of the eluate can be permitted to flow into the analysis channel 4 despite the channel selection.

Aside from the relative angles of the chamber outlet 91, the discard channel 3 and the analysis channel 4 around the junction, other features can be used to implement channel selection.

For example, as shown in FIG. 1, the analysis channel 4 comprises a section 41 adjacent to the channel selector junction 9 that has a smaller cross section than the discard channel 3. This further increases the preferential direction of the eluate into the discard channel 3.

Additionally or alternatively, the discard channel may have a surface or surface coating that is relatively hydrophilic and the analysis channel 4 may have a surface or surface coating that is relatively hydrophobic. For example, the discard channel may have a surface treatment or coating to increase wettability and the analysis channel may have a surface treatment or coating to decrease wettability. In one example, transverse ridges may be provided in the analysis channel to encourage pinning of the liquid meniscus until the discard channel is filled. The discard channel could alternatively or additionally contain an absorbent material which would, e.g., wick the first portion of the eluate. With any of these modifications, the eluate will flow into the hydrophilic channel, and will avoid flowing into the hydrophobic channel until the liquid pressure at the channel selector 9 is sufficient to overcome the repulsive effect of the hydrophobic surface.

A hydrophobic coating may also be used to modify the channel cross section, to simultaneously provide the section 41 with reduced cross-section, to synergistically provide two channel selection effects.

Furthermore, the channel selector 9 may comprise one or more valves or gates, which may be actively controlled to select the desired fraction of the eluate by opening a connection to the analysis channel 4 when the desired fraction is produced.

The discard channel 3 may be a simple channel without any special features, where a length of the discard channel is used to define a volume of the first part of the eluate that is to be discarded. Alternatively, a capacity of the discard channel 3 may be configured by incorporating a wider discard chamber as shown in FIG. 1.

The discard channel 3 can comprise an air outlet 103 and a hydrophobic filter 96 arranged at the air outlet. With this configuration, the discard channel can be initially filled with air. When elution occurs, and the eluate starts flowing through the channel selector 9 and into the discard channel 3, the air flows past the hydrophobic filter 96 and is replaced with the first portion of the eluate. As an alternative to the hydrophobic filter 96, the air outlet may comprise a valve that permits air to flow through, but which closes when the discard channel 3 is filled with eluate and liquid pressure is applied to the valve.

Furthermore, the air outlet 96 can be connected to a closed air chamber 10. The use of a closed air chamber has the benefit that the air pressure increases as the discard channel 3 is filled. This may substantially slow flow of the eluate, and may also be used with a pressure sensor to measure the progress of testing in the cartridge. Additionally, the increased air pressure in the closed air chamber means that the pressure difference across the hydrophobic filter 96 is lower, and thus the stress on the hydrophobic filter 96 is lower.

Returning to the channel selector 9, once discard channel 3 has been filled with an undesired fraction of the eluate, a desired fraction flows into the analysis channel 4. The analysis channel may include various apparatuses for processing and analysing the selected fraction of the eluate.

In the example block diagram of FIG. 1, the analysis channel comprises a lysing section 5 and an amplification section 8. The amplification process carried out in section 8 may, for example, be a thermal cycling process such as PCR or RT-PCR, or may be an isothermal process such as LAMP, or any other biological amplification process. In the example of testing a sample comprising mucin and virus particles, the mucin can act as an inhibitor for the amplification process, and the lysing section 5 may be used to break down virus particles and release viral nucleic acid, and optionally also to deactivate the inhibitor. The amplification section can then be used to amplify the viral nucleic acid for detection.

Both lysing and amplification processes require heating, so the lysing section 5 and amplification section 8 are, e.g., arranged to align with corresponding heaters which may be contained in the cartridge or in an operating module. In some embodiments, to receive heat from the heaters, a wall of the cartridge 1000 separating the fluidic channels from the heaters for the lysing section 5 and amplification section 8 is thin.

To perform even heating of the second portion of the eluate, flow of the eluate in the analysis channel 4 must be controlled. In an embodiment, the second portion of eluate is stopped in the lysing section 5 and then moved on to and stopped in the amplification section 8. This can be achieved by controlling the supply of eluent from the elution source 2.

Additionally, as with the discard channel 3, the analysis channel 4 may comprise an air outlet 107 (and optionally a hydrophobic filter arranged at the air outlet) and connected to a closed air chamber (which may be the same closed air chamber 10 to which the discard channel 3 is connected). Rising air pressure in the not-yet-filled part of the analysis channel 4 may be used to slow the eluate and assist in stopping the eluate.

Furthermore, the analysis channel 4 may comprise one or more valves for actively controlling flow of the eluate.

Alternatively, rather than stopping the second portion of the eluate, the lysing section 5 may receive a continuously flowing second portion of the eluate, and the volume of the lysing section 5 may be configured according to a required heating time and an expected flow rate. In a typical continuous flow examples, the volume of the lysing section 5 is 10-100 μl, the flow rate is 1-10 μl/s and the lysis time is 10-100 s. An example of a combination is a volume of 40 μl, flow rate of 1 μl/s and time of 1 second.

In the example embodiment where the cartridge 1000 is used for analysing a swab sample comprising viral nucleic acid, after RT-PCR amplification is performed using the amplification section 8, a quantity of viral nucleic acid in the second portion of the eluate may be measured. For example, this can be achieved without removing the eluate from the cartridge, for example using known optical characteristics of a marker that binds to the viral nucleic acid. The cartridge 1000, or specifically the amplification section 8, may be constructed from a material which is transparent to a required light wavelength for optical analysis of the second portion of the eluate.

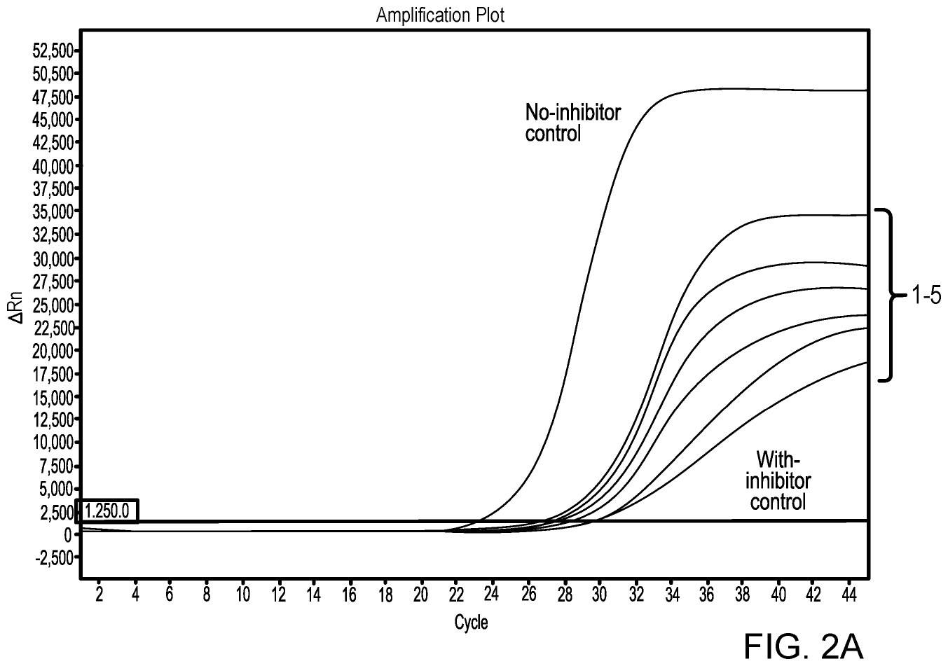

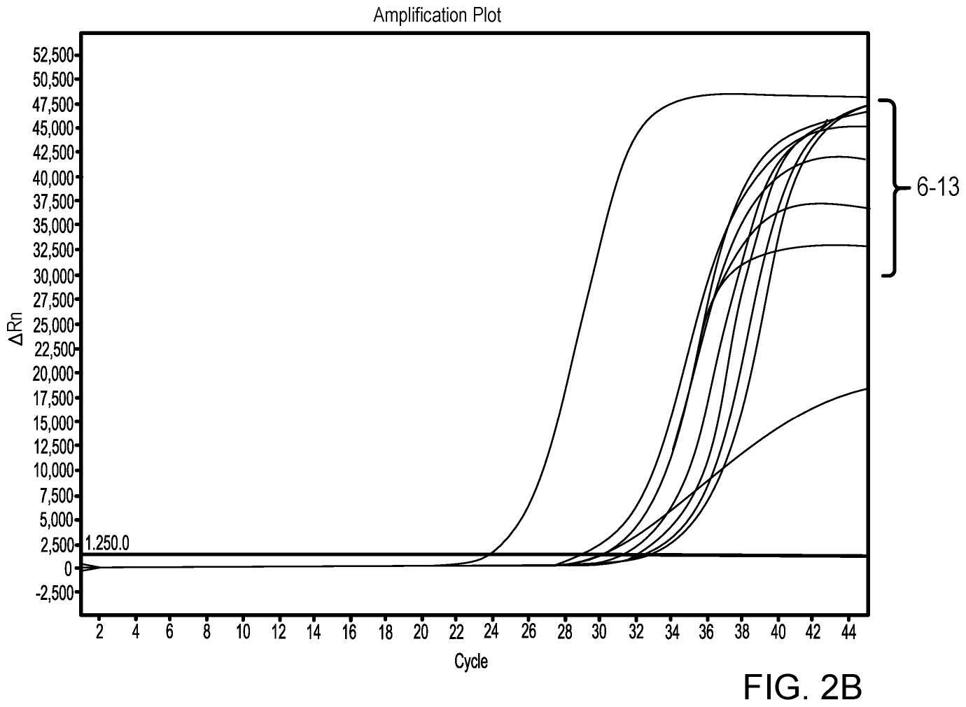

FIGS. 2A and 2B show a specific example of the effects of selecting different fractions of eluate.

More specifically, FIGS. 2A and 2B are PCR amplification curves obtained using different fractions of an eluate.

Each of FIGS. 2A and 2B have uppermost and lowermost curves which are obtained using a conventional technique, where elution is performed separately and fully to obtain a uniform eluate, and then a portion of the uniform eluate is used for lysing and RT-PCR. The uppermost curve is obtained for a solution comprising virus and not comprising mucin or blood. The lowermost curve is obtained for a solution comprising virus, mucin, and blood. As can be seen from these examples, the presence of mucin and blood acts as an inhibitor and reduces the effectiveness of RT-PCR for amplification and detection of the virus specific nucleic acid.

FIG. 2A also shows RT-PCR amplification curves obtained for a first 5 fractions of an eluate produced using the cartridge. As can be seen from FIG. 2A, successive fractions show fluorescence detection above the threshold in fewer amplification cycles and higher end-point fluorescence.

FIG. 2B shows RT-PCR amplification curves obtained for fractions 6 to 13 of an eluate produced using the cartridge. As can be seen from FIG. 2A, while the end-point fluorescence continues to increase in later fractions, the number of cycles taken for the fluorescence signal to cross the detection threshold increases. This is because the later fractions, while having low levels of inhibitor, also have low starting levels of virus, requiring more amplification cycles to generate a detectable product.

From this example, it can be understood that the optimal fraction to select for analysis may be an early fraction, a middle fraction, or a late fraction, depending on the inhibitors and other factors present for the particular type of sample that is being analysed. The optimal fraction can be determined through similar experimentation to assess outcomes for different fractions. Furthermore, in some more complex cases, different testable properties of a sample may appear best in different fractions, such that there are multiple optimal fractions to analyse. Some embodiments can favour later fractions with higher end-point fluorescence signals rather than early fractions with fluorescence detection above the threshold in fewer amplification cycles, to enable robust end-point detection and tolerance of high levels of inhibiting substances.

Once the optimal fraction or fractions has been identified, a channel selector can be configured to select between one or more discard channels and analysis channels using any of the features described above for selecting between channels.

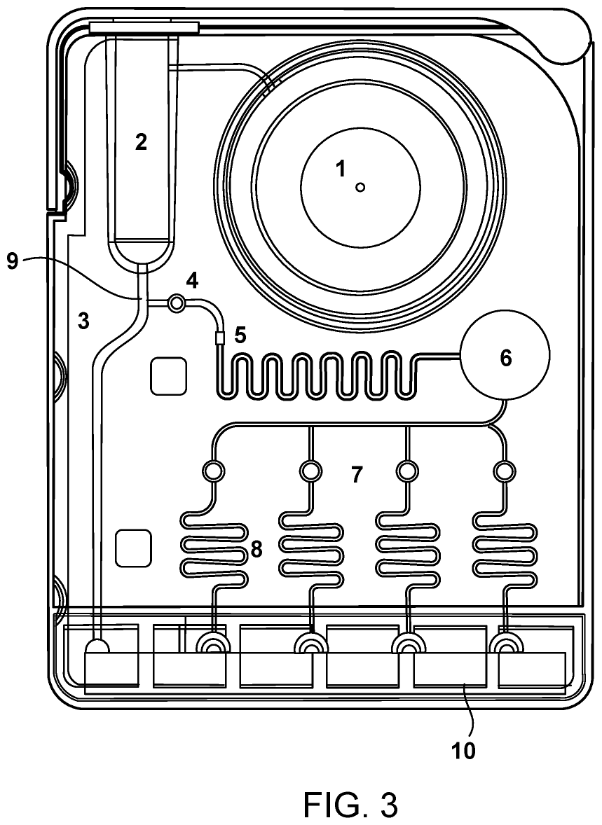

FIG. 3 is a schematic diagram of a specific embodiment of the disposable cartridges described herein. The features of the specific embodiment are largely as described above with reference to FIG. 1, and only specific additional features are described below.

In this example, the channel selector 9 takes the form of a junction where the chamber outlet 91 and the discard channel 3 are arranged substantially opposite each other across the junction 9, and the analysis channel 4 is arranged substantially at a right angle to the chamber outlet 91 around the junction 9. In alternatives, the analysis channel 4 may be arranged, for example, at 30, 60 or degrees to the discard channel 3 around the junction 9.

As shown in FIG. 3, in addition to the elution source 1, the cartridge 1000 may comprise one or more analysis reservoirs 6 comprising additional reagents for analysis. For example, where the analysis channel 4 comprises an amplification section 8, an analysis reservoir 6 may contain enzymes such as reverse transcriptase and polymerase and additional components required for nucleic acid amplification and detection reactions.

Additionally, in this specific embodiment, the lysing section 5 and the amplification section 8 are configured as serpentine channels, such that the second part of the eluate is spread over a heating area without expanding the channel or increasing the complexity of flow beyond the capillary behaviour of a narrow channel.

As also shown in FIG. 3, the amplification section 8 may be split into multiple amplification channels at further junctions 7. The junctions 7 may be configured similarly to the channel selector 9 as described above.

Furthermore, as shown in FIG. 3, the closed air chamber 10 may be arranged outside a plane of the discard channel 3 and the analysis channel 4, to reduce the planar area of the cartridge 1000.

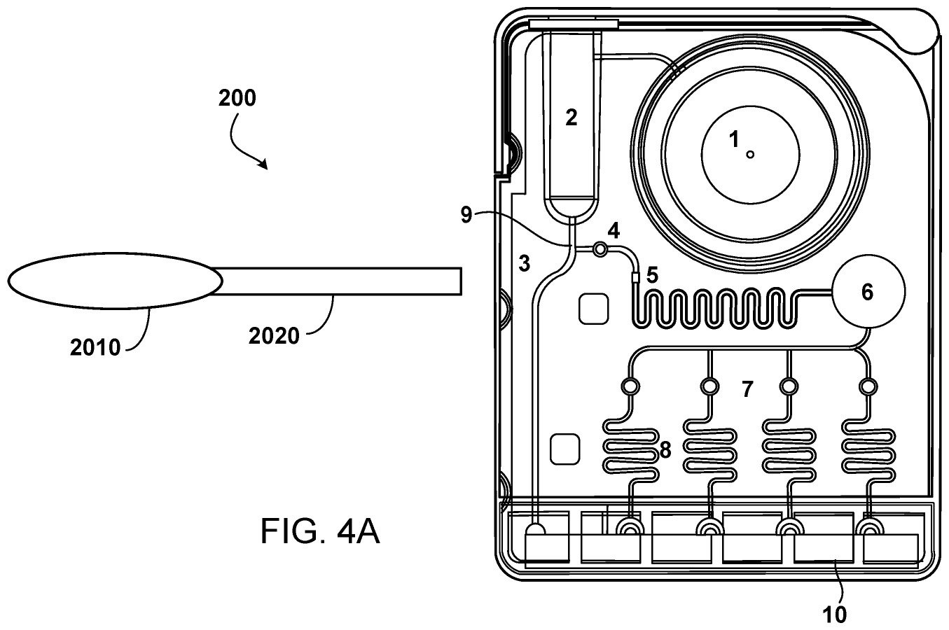

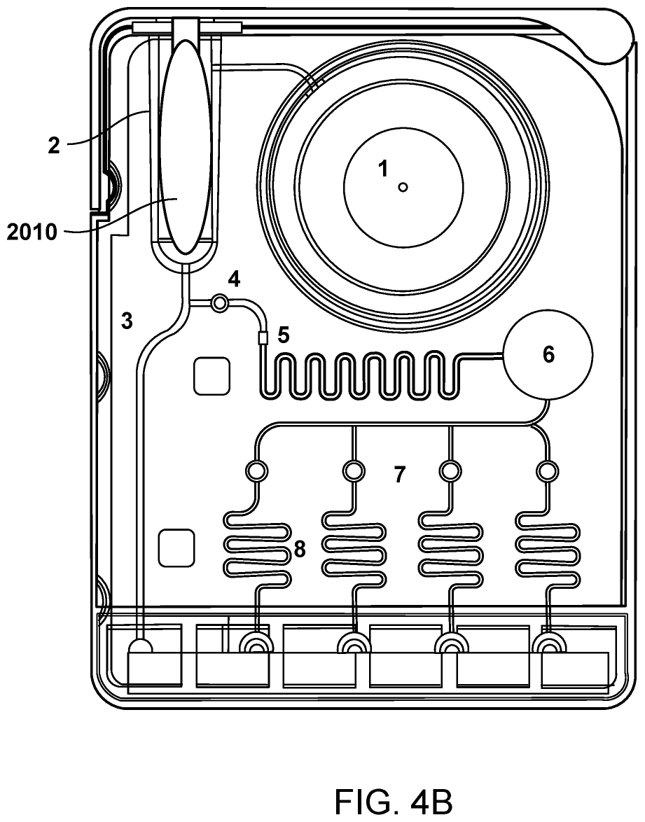

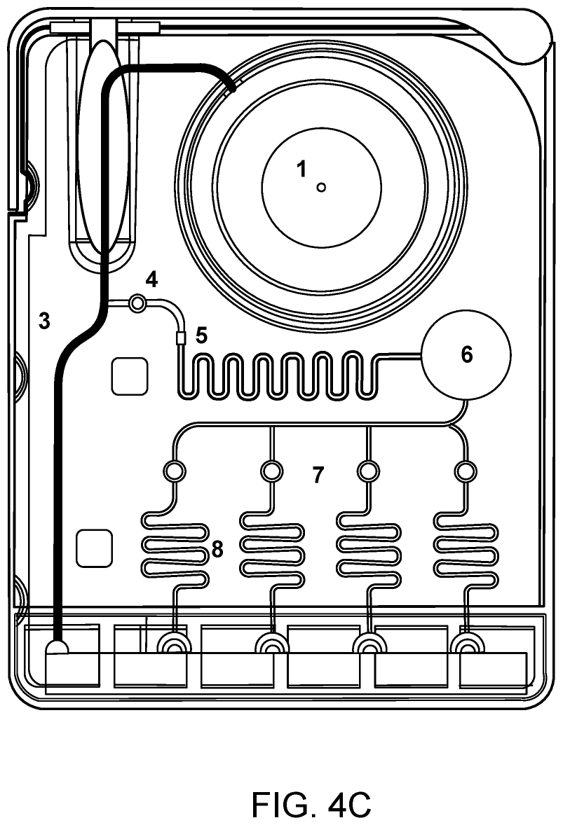

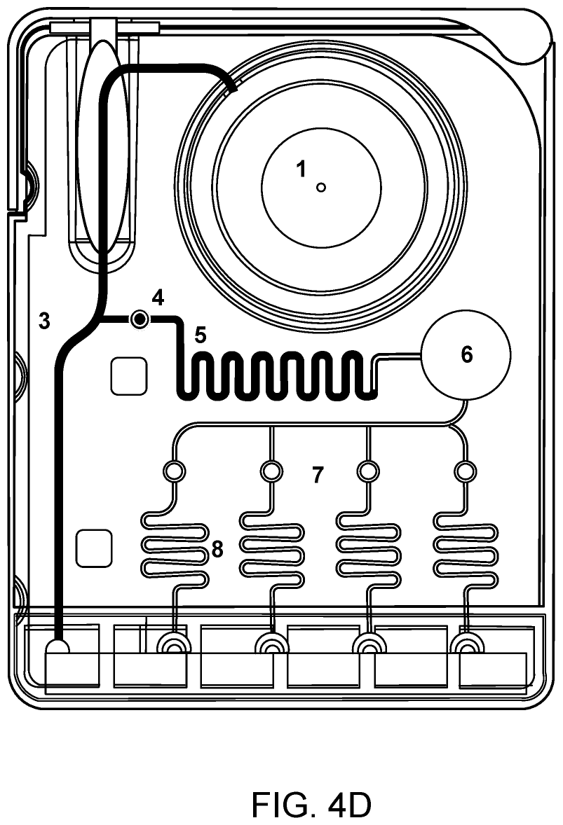

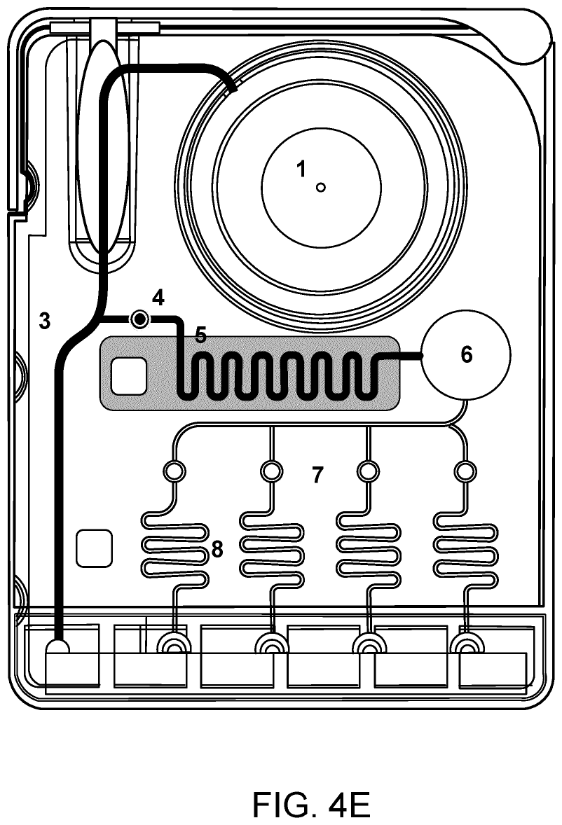

FIGS. 4A to 4E are illustrations of steps in a process of eluting and lysing a sample using a disposable cartridge according to the embodiment of FIG. 3.

In FIG. 4A, an initial state of the cartridge is shown, separate from the swab 2000. The swab 2000 may be a conventional sample swab comprising a head 2010 and a handle 2020. The head 2010 may for example comprise an absorbent section including a material such as polyurethane foam or cotton, polyester, nylon or rayon fibres.

In FIG. 4B, the swab 2000 is arranged the sample chamber 2. In some embodiments, the handle 2020 is configured to detach from the head 2010, so that the sample chamber 2 is substantially filled by the head 2010 and flow of the eluent can be accurately controlled.

In FIG. 4C, the eluent is supplied to the sample chamber 2, and the channel selector 9 directs the first part of the eluate into the discard channel 3.

In FIG. 4D, the discard channel 3 has been filled and the channel selector 9 directs the second part of the eluate into the analysis channel 4 and from there into the lysing section 5.

In FIG. 4E, the lysing section 5 has been filled, and lysing is performed by application of heat.

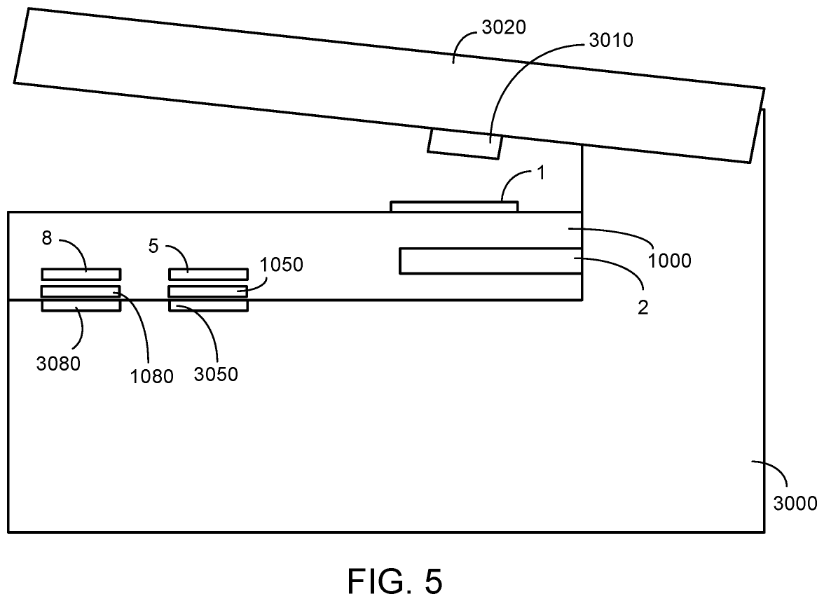

FIG. 5 is a schematic diagram of a sample analysis system in which the cartridge may be used.

Referring to FIG. 5, an operating module 3000 is configured to receive or engage with a cartridge 1000 as described above. For example, the operating module 3000 may have a top-loading configuration in which a lid 3020 is opened to insert the cartridge 1000, or a drawer or slot configuration in which the cartridge is carried or slotted into the operating module.

The operating module 3000 comprises a driver or piston 3010 configured to drive the eluent from the eluent source 1 to the swab chamber 2 within the cartridge 1000. Alternatively, the piston 3010 may be replaced with an external eluent source configured to connect with the elution source 1 of the cartridge 1000. The piston 3010 may, for example, be attached to the lid 3020.

Additionally, in many embodiments, the operating module comprises one or more heater drivers 3050, 3080 arranged to drive corresponding heaters 1050, 1080 contained within the cartridge, to heat eluate in correspondingly arranged sections 5, 8 of the analysis channel 4 in the cartridge 1000.

Alternatively, the operating module itself may comprise one or more heaters arranged to heat the eluate in sections 5, 8.

FIG. 6 illustrates an example of a swab chamber 600 that can be a part of a disposable cartridge as described above. The swab chamber 600 includes an opening 602 configured to receive a swab sample. The swab chamber 600 may also have a lid 603 configured to seal the swab chamber 600. Alternatively, the swab itself may comprise a sealing component to seal the opening, and the lid may be omitted.

When the swab sample is in the swab chamber 600, the swab chamber is sealed and an eluent is run through the swab chamber 600. The swab chamber 600 has an inlet 604 and an outlet 606 for the elution. The inlet 604 is located closest to a tip of the swab head and the outlet 606 is located farthest from the tip of the swab head so that fluid initially impinges on tip of swab head, e.g., where the sample loading is greatest, and flows across the entirety of the swab to the swab outlet 606. This has been qualitatively observed to produce better sample elution than fluid flowing in the opposite direction, i.e., from the swab break point to the tip.

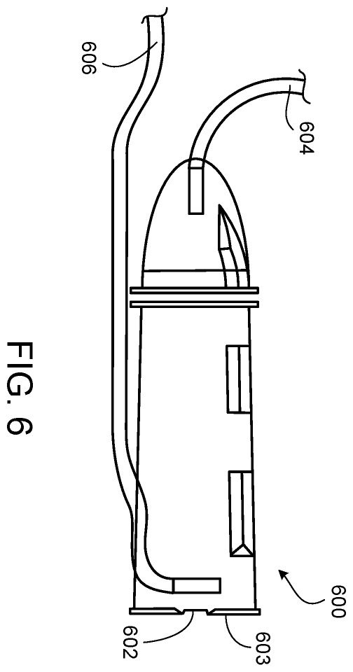

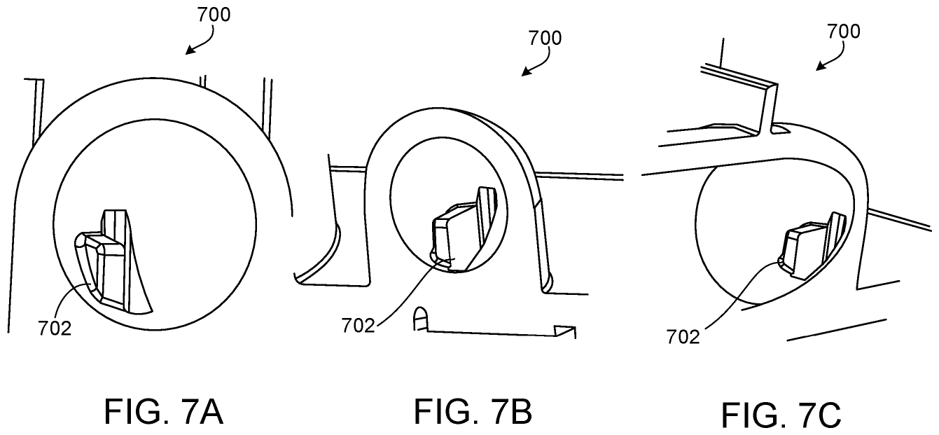

The fluid inlet can include features to prevent a swab inserted into the swab chamber (through a swab port) from being inserted too far or from blocking and sealing the fluid inlet. For example, FIGS. 7A to 7C illustrate perspective views of a fluid inlet 700 that includes a feature 702 (e.g., a projection, protuberance, or ledge) which can prevent a swab from sealing the fluid inlet 700. For example, the feature 702 can contact the tip of the swab and prevent the swab from being inserted into the fluid inlet and sealing the fluid inlet. Keeping the fluid inlet 700 open ensures consistent flow rates and pressures. If the fluid inlet 700 is partially or completely sealed, the flow rates and pressures can be inconsistent and can be problematic for proper elution of the swab. Therefore, it is advantageous to prevent blocking or sealing of the fluid inlet, e.g., with a feature 702.

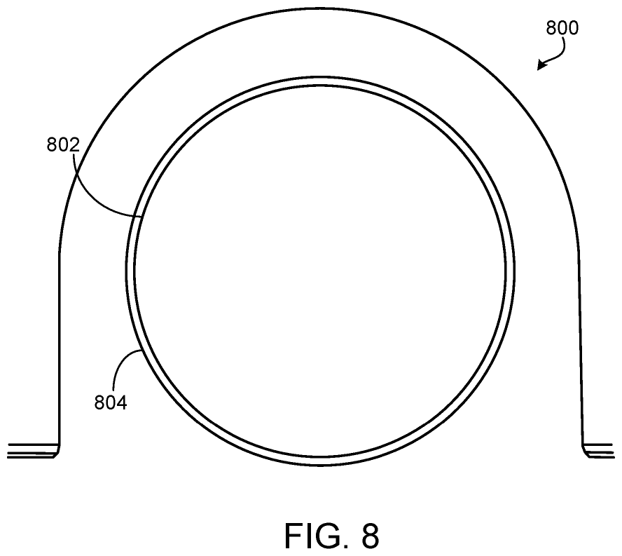

FIG. 8 illustrates a perspective view of a swab chamber 800, which is configured to fit closely around a swab of known size, so that the eluent flows through or closely around the swab to minimize eluent flowing around the outside of the swab head without picking up the sample. For example, a swab chamber can have a cross-sectional area or diameter 802 that is nearly identical to a swab diameter 804. In this way, the swab chamber 800 is ensured to fit tightly around a swab with a swab with a known diameter 804. Additionally, many swabs are partly compressible. For such swabs, the swab chamber may be configured to have a cross-sectional area or diameter 802 that is smaller than the nominal maximum area or diameter of the uncompressed swab 804. Due to the smaller diameter 802, the swab is partly compressed when inserted into the swab chamber 800, and the eluent is forced to pass through the swab (e.g., because it is difficult for the liquid to flow around the swab head) to pick up more of the sample.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A testing device for testing a swab sample, the device comprising:
a swab chamber configured to receive the swab sample;
an elution source configured to supply an eluent to the swab chamber through a chamber inlet of the swab chamber, wherein the eluent is a liquid that interacts with the swab sample to produce an eluate indicative of a property of the swab sample;
a discard channel configured to receive a first portion of the eluate from a chamber outlet of the swab chamber to be discarded;
an analysis channel configured to receive a second portion of the eluate from the chamber outlet to be processed; and
a channel selector configured to selectively direct the first portion into the discard channel and the second portion into the analysis channel.

2. The testing device of claim 1, wherein the chamber inlet is arranged closer to a tip of a swab head than a break point of the swab head and the chamber outlet is located farther from the tip of the swab head than the break point of the swab head, so that the eluent impinges initially on the tip of swab head and flows across the swab head to the chamber outlet.

3. The testing device of claim 1, wherein the chamber inlet comprises a feature that prevents a swab from sealing the chamber inlet.

4. A testing device of claim 1, wherein the elution source comprises a compressible external surface of the testing device.

5. A testing device of claim 1, wherein the channel selector is configured to direct the first portion of the eluate into the discard channel before directing the second portion of the eluate into the analysis channel.

6. A testing device of claim 1, wherein the channel selector comprises a junction between the discard channel, the analysis channel, and the chamber outlet from the swab chamber.

7. A testing device of claim 6, wherein, in the junction, an angle to redirect flow from the chamber outlet to the analysis channel is greater than an angle to redirect flow from the chamber outlet to the discard channel.

8. A testing device of claim 7, wherein the chamber outlet and the discard channel are arranged substantially opposite each other across the junction, and the analysis channel is arranged substantially at a right angle to the chamber outlet around the junction.

9. A testing device of claim 6, wherein the analysis channel comprises a hydrophobic surface.

10. A testing device of claim 1, wherein the discard channel and/or the analysis channel comprises an air outlet and a hydrophobic filter arranged at the air outlet, such that the discard channel and/or the analysis channel can be initially filled with air, and the eluate replaces the air during use.

11. A testing device of claim 10, wherein the air outlet is connected to a closed air chamber.

12. A testing device of claim 1, wherein the analysis channel comprises a lysing section.

13. A testing device of claim 1, wherein the analysis channel comprises an amplification section for amplifying an analyte that is to be detected in the eluate.

14. A testing device of claim 1, wherein the testing device is a disposable cartridge.

15. A testing device of claim 1, wherein an opening of the swab chamber is narrower in diameter than a nominal maximum radius of a swab head.

16. A sample analysis system for testing a swab sample, the system comprising:
a disposable cartridge according to claim 14; and
an operating module for operating the disposable cartridge to test the swab sample,
wherein the operating module is configured to receive or engage with the disposable cartridge, and wherein at least one of:
(i) the operating module comprises an eluent driver configured to drive the eluent from the eluent source to the swab chamber;
(ii) the disposable cartridge comprises a heater for supplying heat to a location in the analysis channel, and the operating module comprises a heater driver arranged to provide electrical drive to the heater; and
(iii) the operating module comprises a heater for supplying heat to a location in the analysis channel.

17. A testing method for testing a swab sample, the method comprising:
arranging a swab sample in a swab chamber;
supplying an eluent to the swab chamber, wherein the eluent is a liquid that interacts with the swab sample to produce an eluate indicative of a property of the swab sample;
directing a first portion of the eluate from the swab chamber to a discard channel to be discarded; and
directing a second portion of the eluate from the swab chamber to an analysis channel to be processed.

18. The testing device of claim 3, wherein the feature comprises a projection, protuberance, or ledge.
